(19) United States
(12) Patent Application Publication
(10) Pub. No.: US 2006/0100908 A1
(43) Pub. Date: May 11, 2006

(54) Matching travel arrangements of medical practitioners with requests for transports of patients

(76) Inventors: Harald Becker, Fluh (CH); Edgar Buhrle, Freiburg (DE)

Correspondence Address: NEIFELD IP LAW, PC, 4813-B EISENHOWER AVENUE, ALEXANDRIA, VA, 22304, US

(21) Appl. No.: 10/974,700

(22) Filed: Oct. 28, 2004

(51) Int. Cl. G06Q 99/00; G06F 19/00

(52) U.S. Cl. 705/3; 705/5; 705/1

(57) ABSTRACT

A method for organizing pre-planned medical assistance for a transport of a patient comprises the computer-implemented step of matching information about travel arrangements of medical practitioners with a request for the transport of the patient, wherein the information about said travel arrangements is contained in at least one computer-implemented database and has been stored in the database prior to the matching step. A computer system and a database comprise corresponding features. The present invention offers an economical way of providing medical assistance for a transport of a patient, and in particular a way by which high-level medical assistance can be organized with little waiting time for the patient.

### FIELD OF THE INVENTION

The present invention concerns the field of organizing pre-planned medical assistance for a transport of a patient.

### BACKGROUND OF THE INVENTION

The steady increase in economic globalization and touristic interest has the consequence that a growing number of people spend a growing amount of time in foreign countries. Therefore a growing number of people can be expected to fall seriously ill while being abroad. Many of these patients wish to be transported back to their respective home countries for medical treatment. Reasons for this wish include language problems, a perception that the medical standard in the respective foreign country is inadequate, problems related to health insurance and the costs for medical treatment in the foreign country, and the desire to be close to relatives and friends.

In a few very serious cases, the transport is only possible in specially equipped, chartered aircraft. However, in the majority of cases, the patient can be transported in the passenger cabin of a common commercial aircraft in the course of a regular scheduled flight. It is imperative in any case that the patient is accompanied by a doctor or another suitably qualified medical practitioner during the transport. This medical practitioner may have to administer certain medications from time to time, and he or she must act immediately if a medical emergency occurs. Experience has shown that, fortunately, such medical emergencies are rare and most of the transports will be uneventful. The services of the medical practitioner are nevertheless called “medical assistance” in the present document, irrespective of whether or not actual medical treatment is required during a particular transport.

A number of companies presently offer services in connection with organizing the transport of patients, including the provision of medical assistance for these transports. The usual practice is that the assistance company receives a request from an insurance company, a hospital or an airline. The assistance company will then arrange the transport by chartering a specially equipped aircraft or by booking seats for the patient and the accompanying doctor on a regular scheduled flight. The seats are usually in the first or business class for allowing the patient to assume a comfortable, reclined position.

The assistance company further organizes the medical assistance by sending a doctor—who may be an employee of the company or a free-lance practitioner who has been retained especially for the transport—to the foreign country from which the transport is to depart. Again, a seat has to be booked for the doctor in an appropriate flight. Depending on the duration of the flight, some rest for the doctor in a hotel may be necessary. All of this adds to the delay and to the costs since the doctor will expect payment for the whole time of the two-way trip.

Irrespective of whether the costs for the transport and the medical assistance are borne by an insurance company or by a travel agency or by the patient himself/herself, a need exists to reduce these costs as much as possible while at the same time providing medical assistance of a high level of quality. Furthermore, a need exists to organize the transport of a patient with little waiting time for the patient.

### OBJECTS AND SUMMARY OF THE INVENTION

It is therefore an object of the present invention to offer an economical way of providing medical assistance for a transport of a patient.

It is a further object of the invention to offer a way of organizing high-level medical assistance for the transport of a patient with little waiting time.

The present invention comprises a method for organizing pre-planned medical assistance for a transport of a patient, the method comprising the computer-implemented step of matching information about travel arrangements of medical practitioners with a request for said transport of said patient, wherein said information about said travel arrangements is contained in at least one computer-implemented database and has been stored in said database prior to said matching step.

The invention further comprises a computer system with at least one database, the computer system being adapted for organizing pre-planned medical assistance for a transport of a patient, the computer system further being adapted for matching information about travel arrangements of medical practitioners with a request for said transport of said patient, wherein said information about said travel arrangements is contained in said at least one database and has been stored in said database prior to said matching.

Yet further, the invention comprises a database with a plurality of travel arrangement records, each of said travel arrangement records containing information about at least one travel arrangement of a medical practitioner, said database being adapted for processing a database query that has been derived from a request for a transport of a patient in order to match information contained in said plurality of travel arrangement records with said request for said transport of said patient.

The invention is based on the practical experience that many medical practitioners enjoy traveling and are among the frequent travelers. Therefore, if medical assistance is required for a transport of a patient within a certain time frame, it is not unlikely that a qualified medical practitioner has already, by chance, booked a place in one of the possible flights. The invention teaches that this—quite probable—coincidence can be used by identifying a suitable medical practitioner who will then be asked to provide the pre-planned medical assistance during the flight.

Using the teaching of the present invention, no costly and time-consuming traveling of the medical practitioner to the place of departure of the flight is necessary. Furthermore, since the medical practitioner intended to take the flight—or at least a similar flight—in any case, it is likely that a low fee can be negotiated with the medical practitioner. The inventors presently assume that many medical practitioners will be willing to provide the medical assistance if they in turn receive a free flight instead of the originally booked flight for which they would have had to pay. A variety of other compensation schemes is also envisaged. For example, the medical practitioner may receive a free upgrade to business or first class and/or bonus points in a frequent flyer program and/or lounge privileges and/or a financial compensation, the amount of which may possibly depend on the actual services that have been necessary in the course of the flight.

In some embodiments of the invention, the request for the transport of the patient forms the basis for a database query in which a matching travel arrangement of a medical practitioner is searched. In other embodiments, both the transport requests and the travel arrangements are stored in the database, and the matching is implemented by a database operation that identifies compatible pairs of travel arrangement data and transport request data. In the latter embodiments, the matching can be performed repeatedly as new travel arrangements are added. Furthermore, it is also possible in these embodiments to review the available transport requests when planning a new travel arrangement. The medical practitioner may thus decide on a travel arrangement for which a corresponding transport request is known to be available.

While in some cases a medical practitioner will prefer to make his or her travel arrangement with a view to the available transport requests, the inventors anticipate that in the majority of cases the travel arrangements will be made for respective purposes that are different from the purpose of providing pre-planned medical assistance for the transport of a patient. This latter constellation of interests has the advantage that the medical practitioner will tend to see any compensation that he or she receives for his or her willingness to provide medical assistance as a welcome added benefit, and not as an integral part of his or her travel arrangements.

In some embodiments, the operation of matching the travel arrangement data and the transport request data is implemented so that the matching will identify at least one travel arrangement that is compatible to at least some degree with the request for the transport of the patient, or so that the matching will identify at least one medical practitioner who has made such a travel arrangement. In this respect, the travel arrangement data and/or the transport request data may, in some embodiments, specify certain allowable ranges, e.g., a time range for the departure or arrival times, or a location range for the acceptable points of departure or arrival.

In some embodiments, an unsharp matching operation may be performed. The unsharp matching operation may assign a certain degree of compatibility to each pair of travel arrangement data and transport request data, depending in how closely the data items match. For example, the unsharp masking operation may assign a medium degree of compatibility to a pair of travel arrangement data and transport request data if the departure time of the travel arrangement data is a few hours different from the optimum departure time given in the transport request data. In such cases, if no better match is found, the medical practitioner or the entity that organizes the transport may be asked whether a changed departure time may be agreed upon.

Preferably, the matching operation takes all relevant information into account, i.e., all information that is necessary for providing the requested transport. This information may include, for example, the availability of capacity for transporting a patient—such capacity is often restricted to a single or a few seats in each airplane—, and any further requirements that may be the consequence of the medical condition of the patient.

The database—which may be a centralized database or a distributed database implemented on a plurality of computers—comprises a large number of database records. In various embodiments of the invention, one or more of the following kinds of records may be present in the database:
records containing information about a particular travel arrangement of a medical practitioner,
records containing information about the respective availability of capacity for transporting a patient in each of a plurality of transport events,
records containing personal information about a medical practitioner,
records containing information relating to the medical equipment available on board during the travel, and
records containing information about a particular request for a transport of a patient.

In preferred embodiments, at least one database is used that contains information about the respective qualifications of the medical practitioners who may be asked to provide their medical services. Such information may comprise, e.g., information about the medical skills and/or the language skills of the respective medical practitioners. The language skills information may identify a native language of the medical practitioner and any other language(s) in which the medical practitioner is competent enough to provide medical assistance. The medical skills may be given in terms of fields of medical specialization—e.g., general practitioners, cardiologists, etc.—and/or in terms of a training level that the medical practitioner completed in a specialized training program.

The specialized training program preferably covers travel-related medical topics. The training program is preferably open at least to the majority or all members of the medical profession. This means that the training is preferably not intended just for a small proportion of doctors (e.g., restricted to doctors that are employed by airlines or assistance companies). To the contrary, it is desirable that as many doctors of all medical fields, languages and qualifications as possible attend the training since this increases the likelihood that at least one qualified doctor can be found whose travel arrangements happen to match a particular transport request.

In some embodiments, the present invention may comprise a portal which allows participating medical practitioners to access necessary or useful information. This information may, for example, be related to the medical equipment available on board of an aircraft. Preferably, the information includes a virtual presentation of the interior of an aircraft and/or a virtual training session with respect to operating medical equipment that is available on board of the aircraft. The medical practitioner accessing this information may select the aircraft he or she is interested in from a choice of types of aircraft, and there may be an automatic pre-selection based on the travel arrangement(s) which the medical practitioner has made.

In many embodiments, the patient will be transported by an aircraft, and consequently the examples given in the present specification mainly concern the transport by aircraft. However, the invention is not limited to aircraft transportation. The invention is also applicable to the organization of medical assistance for the transport of a patient during a ship voyage or any other travel by any means of transportation.

### DETAILED DESCRIPTION OF THE INVENTION

Further features, objects and advantages of the present invention will be apparent from the following detailed description of sample embodiments. Reference is made to the schematic drawings, in which:

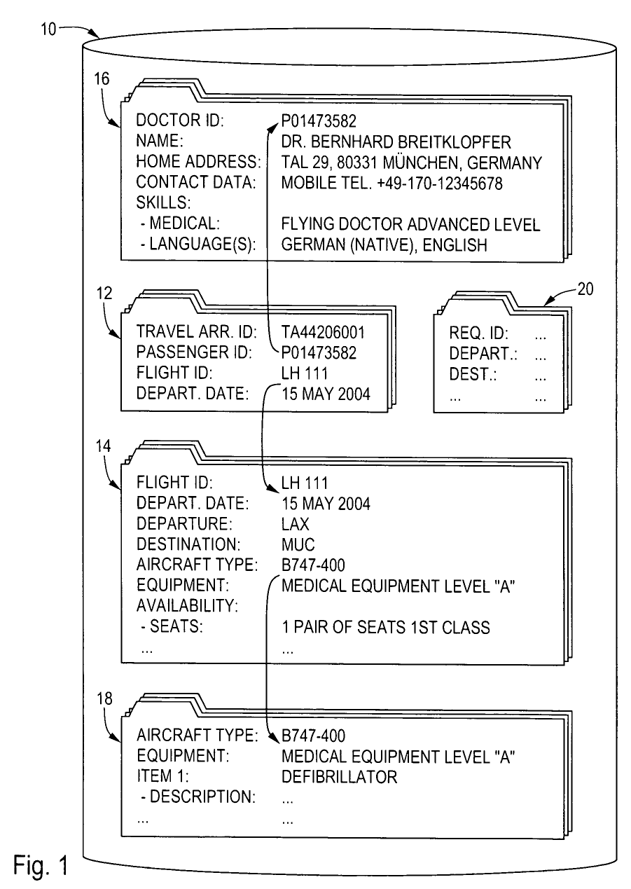

FIG. 1 shows an overview over different kinds of data items stored in a database according to one embodiment of the present invention,

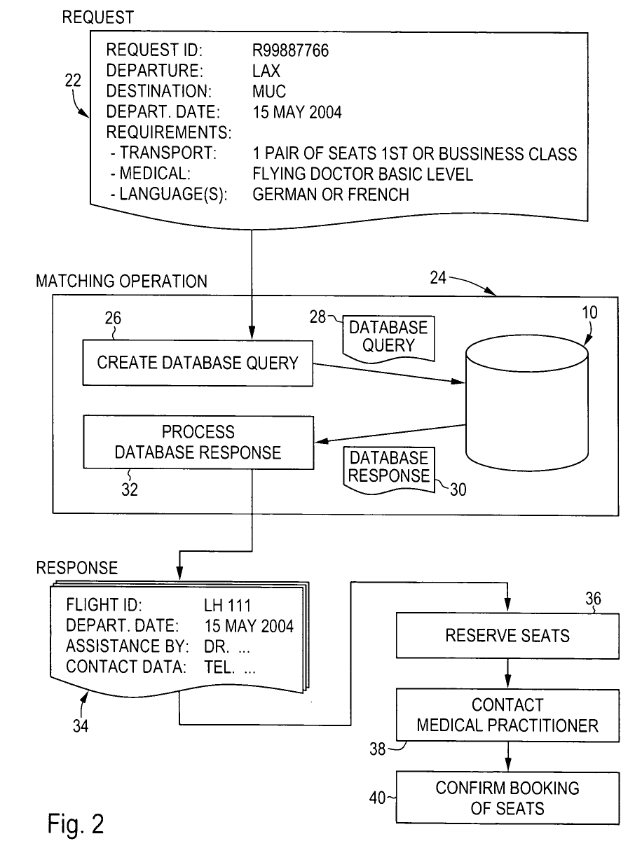

FIG. 2 shows a schematic flow diagram of the process of organizing pre-planned medical assistance using the database of the sample embodiment of FIG. 1, and

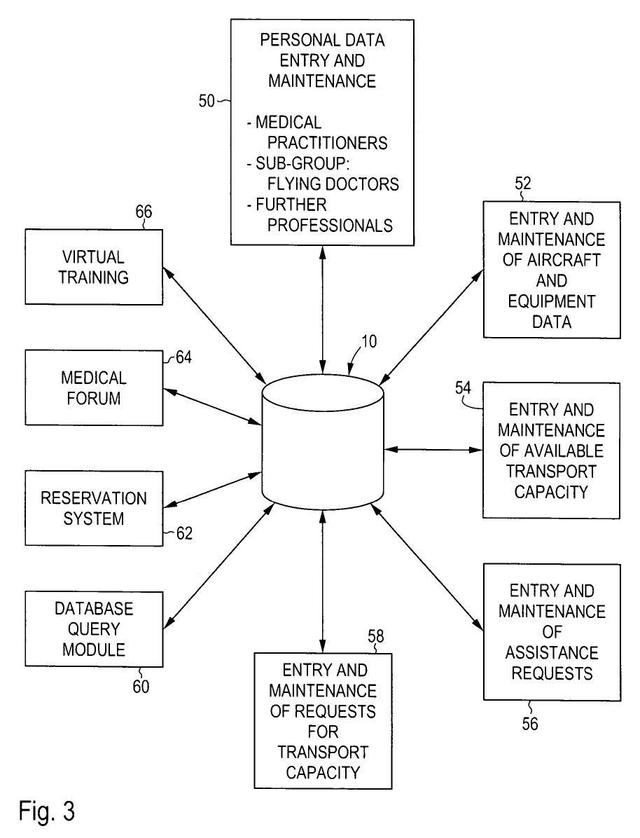

FIG. 3 shows a number of modules for accessing and maintaining data stored in the database.

The sample embodiment shown in FIG. 1 comprises a database 10 with a plurality of records. The term “database” as used herein is intended to encompass any collection of data that is accessible via a computer and/or via a computer network. The database 10 can be implemented by any means either by a stand-alone database application or as a network or cluster or grid of several database management systems running on a number of computers. The computer or computers on which the database 10 is implemented may be of any type, including, e.g., personal computers, workstations, servers and mainframes. These computers may be connected to a local and/or wide-area and/or global computer network.

In the sample embodiment shown in FIG. 1, the database 10 contains a large number of travel arrangement records 12, travel capacity availability records 14, personal information records 16, equipment information records 18, and transport request records 20.

Each travel arrangement record 12 holds information about one particular travel arrangement of a medical practitioner or another participant in the system of the present invention. For example, each travel arrangement record 12 may comprise an identification number of the travel arrangement, an identification number of the passenger, an identification code of the flight, and the planned departure date of the flight.

The flight identification codes and departure dates of the travel arrangement records 12 serve as keys to the travel capacity availability records 14. Each travel capacity availability record 14 contains information about a particular transport event and, in particular, about the availability of transport capacity for this transport event. In the present sample embodiment, each transport event is a flight, but in other embodiments the transport events may be, in addition or instead of flights, sea travels and/or land travels.

In the example of FIG. 1, the information contained in the travel capacity availability records 14 comprises the flight identification code, the departure date, the airport abbreviations of the departure and destination airports, the aircraft type, a code summarizing the medical equipment available on board of the aircraft, and the current availability of seats for patient transports. It is apparent that further information, e.g., details about the facilities offered for the patient transports, may also be contained in the travel capacity availability records 14.

In the present sample embodiment, each of the travel capacity availability records 14 concerns a single transport event. However, alternative embodiments are envisaged in which each travel capacity availability record 14 specifies data that is common to a plurality of transport events, e.g., data that is common to all flights that have a certain identification code.

Returning to the example of FIG. 1, the passenger identification of the travel arrangement records 12 serves as a key to the personal information records 16. Each personal information record 16 contains personal information of a medical practitioner who has been registered as a participant in the system of the present invention. In the present sample embodiment, the personal information includes an identification number (corresponding to the passenger identification of the travel arrangement records 12), personal identifying information of the medical practitioner (e.g., his or her name and home address), foreign contact data of the medical practitioner (e.g., a mobile telephone number and/or an email address and/or a foreign address under which the medical practitioner can be contacted), and information about the respective skills of the medical practitioner in a number of categories.

The categories in which the skills of the medical practitioners are given in the personal information records 16 may include, e.g., medical skills and language skills. The data contained in the language skills category may comprise the native language of the medical practitioner and any other language(s) in which he or she is proficient enough for communicating with a patient. The medical skills may be given in one or both of the following aspects: First, one or more medical specializations of the medical practitioner (e.g., cardiologist, neurologist, . . . ) may be stated. Second, it may be stated whether or not the medical practitioner has attended and successfully completed a specialized training. This training, which will be called “Flying Doctor training” in the following, is intended to provide a medical practitioner with comprehensive knowledge and skills in the fields of emergency and travel related medicine. In some embodiments, several training levels (e.g., basic, advanced, . . . ) may be distinguished.

The Flying Doctor training comprises a review of generally approved techniques for the in-flight handling of medical emergencies, a familiarization with the medical equipment that is available on board, and a communication training. The training comprises a theoretical part as well as a practical part in which typical cases are simulated, together with cabin crew members, in a cabin simulator or an aircraft simulator or on board of an aircraft.

Besides the basic training covering fundamental issues, there may be specialized courses for particular problems and brush-up courses for renewing the doctors' knowledge and skills. The Flying Doctor training may also comprise information about the legal situation of doctors when they provide medical assistance on board of an aircraft. Furthermore, the doctors may be given the opportunity to sign a pre-negotiated contract with an assistance provider or an airline.

Further details of the Flying Doctor training in a possible embodiment of the present invention are described in U.S. patent application Ser. No. 10/832,364, which has been filed on Apr. 27, 2004, by the inventors of the present invention. The contents of this earlier application are herewith incorporated into the present specification in their entirety.

Returning to the database 10 shown in FIG. 1, the contents of the aircraft type and equipment fields of the travel capacity availability records 14 serve as keys for the equipment information records 18. For each aircraft type and medical equipment package, there is a corresponding equipment information record 18 that lists the individual pieces of equipment, together with a detailed description (including, e.g., directions for use of the equipment). The record 18 may also comprise links to further information, e.g., to multimedia content that may include an instruction manual or a video demonstration showing the use of the available equipment.

The database 10 shown in FIG. 1 further comprises a plurality of transport request records 20. Each transport request record 20 contains information corresponding to a request for providing medical assistance for a transport of a patient. This information will be described in more detail in the discussion of FIG. 2 below. In the present embodiment, a transport request record 20 is stored for each transport request that cannot be fulfilled immediately. Whenever a new travel arrangement record 12 is added to the database 10, a matching operation can be performed in order to identify any open transport requests that might possibly match with this new travel arrangement record 12. In alternative embodiments, all transport requests are stored as transport records 20 in order to provide a full history of any data that has been entered into the system. In yet further alternative embodiments, no transport request records 20 are stored in the database 10.

It is apparent that the structure of the database 10 shown in FIG. 1, including the particular kinds of records and information contained therein, represents only a sample embodiment of the present invention. Many alternative data arrangements are possible and are contemplated by the inventors.

FIG. 2 depicts a sequence of steps in which, according to a sample embodiment of the present invention, a request 22 for a transport of a patient is matched in a matching operation 24 with the contents of the database 10. In the embodiment shown in FIG. 2, the request 22 contains a request identification, the airport codes for the departure and destination airports of the transport flight, the requested departure date (or a selection or range of dates), and the requirements for the transport. These requirements may include, for example, transport requirements (e.g., the number and quality of seats that are required), medical requirements (e.g., the minimum required flying doctor training level and/or any other medical qualifications), and language requirements (e.g., a choice of languages with the requirement that the medical practitioner must be proficient in at least one of these languages). It is apparent that additional or different or less data items may be contained in the request 22 in alternative embodiments.

In the present sample embodiment, the data items of the request 22 are entered by an operator of an assistance provider into corresponding data entry fields of a computer screen mask. The request 22 is then processed immediately in the matching operation 24. Optionally, the request 22 may be stored in the database 10 as a transport request record 20. In the present sample embodiment, the data contained in the transport request record 20 (FIG. 1) is exactly identical to the data contained in the request 22 (FIG. 2). Different data formats may be used in alternative embodiments. For example, the request identification may be absent from the request 22, and it may only be generated automatically when storing the request 22 in a transport request record 20.

The matching operation 24 is performed by a computer system that may house the database 10 or communicate with the database 10 via a local or wide-area or global network. In a first step 26, a database query 28 is generated from the request 22. The database query 28 may comprise database instructions in any database instruction code (e.g., the structured query language SQL) that can be processed by the database 10. The database query 28 comprises code that effects a matching of the requirements contained in the request 22 with the contents of the database 10, in particular, with the travel arrangement records 12 and the travel capacity availability records 14.

It is apparent that for some data items (e.g., the departure and destination airports) an exact match is preferable. Other data items in the request 22 (e.g., the medical qualifications) only specify minimum requirements which match perfectly if “the same or better” conditions are available. Yet further data items in the request 22 (e.g., a range of departure dates or a choice of acceptable language skills) offer a variety of possible matchings. The database query 28 is generated in step 26 in such a way that all of these cases are taken into account.

The above examples concerned exact matchings, i.e., matchings that fully meet the requirements stated in the request 22. In some embodiments of the invention, a mechanism for performing an unsharp matching operation is also provided. The unsharp matching operation may identify various degrees of compatibility between the request 22 and the entries in the database 10. For example, the unsharp matching operation may identify an otherwise suitable flight that departs not from the departure airport specified in the request 22 but from a nearby airport. Depending on the distance between these airports, a certain degree of compatibility (e.g., 80%) may be assigned to the matching.

After processing the database query 28 using methods that are known as such in the database arts, the database 10 outputs a database response 30. The database response 30 is processed in step 32 in order to yield one response 34 or a plurality of responses 34 in answer to the request 22. Each response 34 preferably contains all information that is necessary to further process the request 22.

In the present embodiment, the information given in the response 34 (or each response 34) contains the flight identification and departure date gathered from a matching travel arrangement record 12 and a matching travel capacity availability record 14. The response 34 further contains the name and contact data of the medical practitioner gathered from the personal information record 16, as indexed by the passenger identification stated in the matching travel arrangement record 12.

Further responses 34 are generated if there is more than one perfect match or if an unsharp matching operation is performed; in the latter case, the responses 34 are preferably ordered according to their respective degrees of compatibility. It is apparent that different or additional or fewer pieces of information can be given in the response 34 in alternative embodiments.

If there is more than one response 34, the operator of the assistance company picks the most appropriate one. The operator will then tentatively reserve, in step 36, the required number of seats and any other necessary resources in the travel event specified in the response 34. Then the operator contacts, in step 38, the medical practitioner at his or her contact data as specified in the response 34. This step can be performed automatically if the contact data is an email address or if a synthesized voice system is used to call the practitioner at his or her telephone number.

As soon as the practitioner has confirmed that he or she will be available to provide medical assistance, the booking of the required number of seats for the patient and the medical practitioner for the respective flight is confirmed in step 40. All necessary travel arrangements have thus been completed with a minimum of effort, cost and waiting time for the patient.

While a specific example of the use of the database 10 has been described above in detail with respect to FIG. 1 and FIG. 2, the database 10 of the present invention is generally intended to be a global database for the management of all resources that concern medical issues in travel-related businesses. All information items and resources that are required for medical care and medical assistance during travel events are contained in the database 10 and may be accessed by authorized companies and individuals all over the world.

Summing up some of the functions of the database 10, medical practitioners, who may have completed the Flying Doctor training, are recorded in the database 10 together with information about their respective qualifications. Airlines enter information about their aircraft fleets and about the available capacity for assistance flights into the database 10. Requests for transports of patients and information about the availability of medical practitioners may be accessed using the database 10. Assistance providers enter their requests for transports into the database 10 and access the data of the airlines and medical practitioners. Further functions of the database 10 include a virtual training that can be used by the medical practitioners to make a virtual tour through an aircraft and the available medical equipment. The virtual training can also be used to practice a simulated (virtual) emergency with the assistance of a hotline.

An important further advantage of the database 10 is that it may be accessed for identifying medical practitioners that may happen to be on board of an aircraft if a medical emergency occurs during a flight. The database 10 may also be used to verify the identity of any person that purports to be a qualified medical practitioner in response to an in-flight emergency announcement. In other words, the database 10 of the present invention can be used as the database that is mentioned in U.S. patent application Ser. No. 10/832,364, which has been filed on Apr. 27, 2004, by the inventors of the present invention. The contents of this earlier application are herewith incorporated into the present specification in their entirety.

FIG. 3 schematically shows a number of modules that interact with the database 10 (or, in some embodiments of the invention, are part of the database 10). The modules provide suitable front-end functionality for the database 10, in particular for entering, managing and accessing data contained in the database 10. It is apparent that in various embodiments of the present invention, different or fewer or additional modules than those shown in FIG. 3 may be present.

A first module 50 concerns the entry and maintenance of personal data. Generally, all medical practitioners that have an interest in travel-related medicine may be recorded in the database 10, together with their respective contact data. The participants receive the option of making their availability known in response to on-line requests. This makes it possible to identify a suitable medical practitioner for providing medical assistance in the manner described above.

A sub-group within the overall group of medical practitioners are doctors that have successfully completed the above-described Flying Doctor training. Additional data may be entered for such persons, e.g., data regarding the attained Flying Doctor training level or other information that is voluntarily given by the doctor.

In addition to the above-mentioned medical practitioners, further professionals or organizations that can provide medical and/or travel related services may be recorded in the database 10 using functions provided by the first module 50.

All persons that are recorded in the database 10 have the option to control to what extent their personal data is allowed to be used. For example, each person may decide whether or not his or her personal data is allowed to be published, and whether or not the data may be accessed by airline carriers or assistance companies. The data may also be used, if desired, to provide the medical practitioners or only the qualified Flying Doctors or any other sub-group with relevant information.

A second module 52 concerns the entry and maintenance of aircraft and equipment data. This module 52 is used to enter the various types of aircraft and the available medical equipment as well as the respective locations within each aircraft wherein the equipment is stored. Furthermore, the seats or groups of seats that are cleared for assistance flights may be entered into the database 10 using the module 52, together with any relevant details as requested by the assistance companies.

A third module 54 offers functions for entering and updating the available capacity for assistance transports in each flight. This module is used by the airlines and transport companies, and it may be connected on-line with the reservation systems of the airlines. All pieces of information that are relevant for providing high-quality medical assistance are contained in the database 10.

A fourth module 56 is used for entering and updating the requests for medical practitioners and services in the field of travel-related medicine, in particular the requests for medical assistance. This module processes and stores the requests. Possible users of this module are assistance companies, travel agencies, hospitals, emergency rooms, insurance companies, and so on.

The requests for transport capacity are entered and updated by means of a fifth module 58. Assistance companies use this module 58 to enter their requirements in order to find the most suitable offer among the available transport events.

A sixth module 60 enables the users and operators to conduct database queries regarding all resources managed in the database 10. The database queries can be performed in a dialog-supported fashion. A function is provided to verify the availability of a resource after the resource has been selected.

A seventh module 62 shown in FIG. 3 implements a reservation system that can be used to reserve or book the resources that are managed within the database 10. The reservation system will normally be connected directly to the respective reservation systems of the airlines or other participants. An immediate confirmation of any reservation is possible.

An eighth module 64 shown in FIG. 3 provides a medical forum for airlines and medical practitioners for distributing information and news relevant to the participants.

Finally, a ninth module 66 provides sophisticated virtual training functions. For example, participants of Flying Doctor training courses or other interested persons may re-train certain lessons, thus refreshing and re-affirming their skills and knowledge. The virtual medical training may also comprise an introduction to new methods and new medical equipment, with the possibility to virtually operate the medical equipment.

Another function of the virtual training module 66 is to offer a virtual tour through an aircraft of a particular type. This tour may be useful for the medical practitioner to familiarize himself or herself with the particulars of the respective type of aircraft and the location and any special features of the medical equipment available therein. Yet another function of the virtual training module 66 is that medical and other equipment (e.g., defibrillators, ECG equipment, communication equipment, and so on) is presented for virtual operation and for virtual training. Furthermore, the virtual training module 66 may offer a function in which emergency situations are simulated in real-time. This emergency training may be controlled remotely by a medical hotline, and it may include data transmission to the hotline and simulated responses by the hotline.

It is apparent that the modules shown in FIG. 3 and described above only represent some examples of modules and functions that are offered by the database 10 or can be implemented in connection with the database 10. Many other functions, or variations of the functions described above, are possible and will be obvious to the person skilled in the art. Furthermore, it is apparent that not all of the modules described above need to be present in order to provide a working and efficient system.

All in all, the present invention provides a way for matching travel arrangements of medical practitioners with requests for transports of patients that combines a high level of medical service with good economy and little waiting time for the patients. In preferred embodiments and further developments, a comprehensive database system with a variety of uses in the field of travel-related medical services is provided.

The particulars contained in the above description of sample embodiments should not be construed as limitations of the scope of the invention, but rather as exemplifications of preferred embodiments thereof. Accordingly, the scope of the invention should be determined not by the embodiments illustrated, but by the appended claims and their legal equivalents.

We claim:

1. A method for organizing pre-planned medical assistance for a transport of a patient, the method comprising the computer-implemented step of matching information about travel arrangements of medical practitioners with a request for said transport of said patient, wherein said information about said travel arrangements is contained in at least one computer-implemented database and has been stored in said database prior to said matching step.

2. The method of claim 1, wherein said medical practitioners have made their respective travel arrangements for respective purposes that are different from the purpose of providing said pre-planned medical assistance for said transport of said patient.

3. The method of claim 1, wherein said matching step is adapted to identify at least one of:
a travel arrangement that is compatible at least to some degree with said request for said transport of said patient, and
a medical practitioner whose travel arrangements are compatible at least to some degree with said request for said transport of said patient.

4. The method of claim 1, wherein said database comprises a plurality of travel arrangement records, each of said travel arrangement records containing information about at least one travel arrangement of a medical practitioner.

5. The method of claim 1, wherein said matching step includes processing of a database query that has been derived from said request for said transport of said patient.

6. The method of claim 1, wherein an unsharp matching operation is performed that assigns degrees of compatibility to a plurality of pairs of travel arrangement data and transport request data.

7. The method of claim 1, wherein information about the respective availability of capacity for transporting a patient in each of a plurality of transport events is taken into account in said matching step.

8. The method of claim 7, wherein each transport event is one of an aircraft flight and a ship voyage.

9. The method of claim 7, wherein said at least one database comprises a plurality of travel capacity availability records, each of said travel capacity availability records containing information about the respective availability of capacity for transporting a patient in each of said plurality of transport events.

10. The method of claim 1, wherein said at least one database further contains information concerning the qualifications of each medical practitioner recorded therein.

11. The method of claim 1, wherein said at least one database comprises a plurality of personal information records, each of said personal information records containing personal information about a medical practitioner.

12. The method of claim 1, wherein only medical practitioners who have completed a special training related to travel-related medical skills are allowed to provide said pre-planned medical assistance.

13. The method of claim 12, wherein said special training is intended for at least the majority of all members of the medical profession.

14. The method of claim 1, wherein said at least one database comprises a plurality of equipment information records, each of said equipment information records containing information relating to the medical equipment available on board of at least one type of aircraft.

15. The method of claim 1, wherein participating medical practitioners are provided with access for obtaining information related to the medical equipment available on board of at least one type of aircraft.

16. The method of claim 15, wherein said information is provided in the form of one of:
a virtual presentation of the interior of an aircraft of said type of aircraft, including the location of the medical equipment available on board of said aircraft, and
a virtual training session with respect to operating the medical equipment available on board of an aircraft of said type of aircraft.

17. The method of claim 1, wherein said at least one database comprises a plurality of transport request records, each of said transport request records containing information about at least one request for a transport of a patient.

18. The method of claim 1, wherein said travel arrangements concern aircraft flights.

19. The method of claim 1, wherein said travel arrangements concern sea travels.

20. A computer system comprising at least one database, the computer system being adapted for organizing pre-planned medical assistance for a transport of a patient, the computer system further being adapted for matching information about travel arrangements of medical practitioners with a request for said transport of said patient, wherein said information about said travel arrangements is contained in said at least one database and has been stored in said database prior to said matching.

21. The computer system of claim 20, wherein said medical practitioners have made their respective travel arrangements for respective purposes that are different from the purpose of providing pre-planned medical assistance for said transport of said patient.

22. The computer system of claim 20, wherein said matching is adapted to identify at least one of:
a travel arrangement that is compatible at least to some degree with said request for said transport of said patient, and
a medical practitioner whose travel arrangements are compatible at least to some degree with said request for said transport of said patient.

23. A database comprising a plurality of travel arrangement records, each of said travel arrangement records containing information about at least one travel arrangement of a medical practitioner, said database being adapted for processing a database query that has been derived from a request for a transport of a patient in order to match information contained in said plurality of travel arrangement records with said request for said transport of said patient.

24. The database of claim 23, wherein the travel arrangements have been made for respective purposes that are different from the purpose of providing said pre-planned medical assistance for said transport of said patient.

25. The database of claim 23, wherein said matching is adapted to identify at least one of:
a travel arrangement that is compatible at least to some degree with said request for said transport of said patient, and
a medical practitioner whose travel arrangements are compatible at least to some degree with said request for said transport of said patient.

26. The database of claim 23, wherein said database comprises a plurality of travel capacity availability records, each of said travel capacity availability records containing information about the availability of capacity for transporting a patient in at least one transport event, said database being adapted for taking into account information contained in said plurality of travel capacity availability records in said matching.

27. The database of claim 26, wherein said transport event is one of an aircraft flight and a ship voyage.

28. The database of claim 23, wherein said database comprises a plurality of personal information records, each of said personal information records containing personal information about a medical practitioner.

29. The database of claim 28, wherein said personal information about said medical practitioner comprises information concerning the qualifications of said medical practitioner.
